(19) United States
(12) Patent Application Publication
(10) Pub. No.: US 2020/0253976 A1
(43) Pub. Date: Aug. 13, 2020

(54) Endothelial Facilitation in Neurodegerative Diseases by Cerebral Blood Flow Enhancement

(71) Applicant: UNIVERSITY OF MASSACHUSETTS, Boston, MA (US)

(72) Inventor: David Drachman, Concord, MA (US)

(21) Appl. No.: 16/651,570

(22) PCT Filed: Sep. 28, 2018
(86) PCT No.: PCT/US18/53324

§ 371 Date: Mar. 27, 2020

Related U.S. Application Data
(60) Provisional application No. 62/564,615, filed on Sep. 28, 2017.

(51) Int. Cl. A61K 31/519; A61K 31/155; A61K 31/17; A61K 31/40; A61K 31/405; A61K 31/366; A61K 31/47; A61K 31/25; A61K 31/505; A61P 25/28; A61K 45/06

(52) U.S. Cl. CPC A61K 31/519; A61K 31/155; A61K 31/17; A61K 31/40; A61K 31/405; A61K 45/06; A61K 31/47; A61K 31/25; A61K 31/505; A61P 25/28; A61K 31/366

(57) ABSTRACT

The “amyloid hypothesis” has dominated Alzheimer research for more than 20 years, and proposes that amyloid is the toxic cause of neural/synaptic damage and dementia. Despite discrepancies in the proposed mechanism, and failed clinical trials, amyloid continues to be considered the cause of a degenerative cascade. The present invention proposes that AD is precipitated by impaired microvascular function, resulting primarily from decreased Notch-related angiogenesis. With impaired microvasculature, a lack of vascular endothelial-derived trophic factors and decreased cerebral blood flow cause the atrophy of neural structures. Therapeutic strategies are proposed that focus on supporting normal angiogenesis.

### FIELD OF THE INVENTION

The present invention is in the field of neurodegenerative diseases. In particular, a treatment regimen including, but not limited to, including simvastatin, L-arginine, and/or tetrahydrobiopterin is identified that improves cognitive function in patients diagnosed with a neurodegenerative disease (e.g., Alzheimer's disease). Such drugs may improve cerebral microvascular endothelial function by introducing progenitor endothelial cells into the cerebral microvascular circulation. Alternatively, the proposed mechanism is consistent with the use of endothelin-1 inhibitors to prevent, arrest or modify the progression of AD and related disorders.

### BACKGROUND

Currently, more than five (5) million people in the United States and thirty-five (35) million throughout the world currently have Alzheimer's disease (AD). Billions of dollars have already been spent by pharmaceutical companies in an effort to find a treatment that favorably alters the course of AD.

There are at present no treatments that significantly modify the course of Alzheimer's disease (AD). For example, four (4) drugs have been approved by the United States Food & Drug Administration of which three (3) are anticholinesterases and one (1) is an N-methyl-D-aspartate (NMDA) receptor blocker. It is believed that these drugs only modify the symptoms of memory and cognitive impairment that occur with AD, but do not alter the progression of the disease or the mechanism by which it causes neuronal losses and dementia. Many drugs are under study in an effort to slow, arrest or reverse the course of AD. The majority of these potential therapies attempt to interfere with the formation of beta amyloid, or remove it from the brains of affected individuals. So far, all such drugs have failed to modify the course of AD favorably.

What is needed in the art is a therapeutic approach that does not directly affect the beta-amyloid plaque formation, but can reduce the risk of dementia and improve brain function in patients who have developed Alzheimer's disease and other related degenerative disorders through indirect mechanisms that improve the overall health of the brain.

### SUMMARY OF THE INVENTION

The present invention is in the field of neurodegenerative diseases. In particular, a treatment regimen including, but not limited to, including simvastatin, L-arginine, and/or tetrahydrobiopterin is identified that improves cognitive function in patients diagnosed with a neurodegenerative disease (e.g., Alzheimer's disease). Such drugs may improve cerebral microvascular endothelial function by introducing progenitor endothelial cells into the cerebral microvascular circulation. Alternatively, the proposed mechanism is consistent with the use of endothelin-1 inhibitors to prevent, arrest or modify the progression of AD and related disorders.

In one embodiment, the present invention contemplates a method, comprising: a) providing; i) a patient exhibiting at least one symptom of a neurodegenerative disease, and ii) a pharmaceutical composition comprising a statin; and b) administering said pharmaceutical composition to said patient under conditions such that said at least one symptom is reduced. In one embodiment, the pharmaceutical composition further comprises a nitric oxide synthase substrate. In one embodiment, the pharmaceutical composition further comprises a biopterin compound. In one embodiment, the pharmaceutical composition comprises a combination of said statin and a nitric oxide synthase substrate. In one embodiment, the pharmaceutical composition comprises a combination of said statin, said nitric oxide synthase substrate and a biopterin compound. In one embodiment, said statin pharmaceutical composition is administered for a first time period. In one embodiment, said combined statin/nitric oxide substrate pharmaceutical composition is administered for a second time period. In one embodiment, said combined statin/nitric oxide substrate/biopterin compound composition is administered for a third time period. In one embodiment, said first time period precedes said second time period. In one embodiment, said second time period precedes said third time period. In one embodiment, said first time period is one month. In one embodiment, said second time period is one month. In one embodiment, said third time period is two months. In one embodiment, said statin includes, but is not limited to, atorvastatin, fluvastatin, lovastatin, pitavastatin, pravastatin, rosuvastatin and/or simvastatin. In one embodiment, said nitric oxide substrate includes but is not limited to, L-arginine, L-citrulline and/or NG-hydroxy-L-arginine. In one embodiment, the biopterin includes, but is not limited to, tetrahydrobiopterin and/or dihydrobipterin. In one embodiment, said at least one symptom comprises reduced cognitive function. In one embodiment, said at least one symptom comprises cerebral atrophy. In one embodiment, said at least one symptom comprises reduced brain microvascular endothelial function. In one embodiment, said combined statin/nitric oxide substrate pharmaceutical compound is synergistic as compared to said statin pharmaceutical compound. In one said combined statin/nitric oxide substrate/biopterin compound pharmaceutical compound is synergistic as compared to said statin pharmaceutical compound. In one embodiment, said combined statin/nitric oxide substrate/biopterin compound pharmaceutical compound is synergistic as compared to said combined statin/nitric oxide substrate pharmaceutical compound. In one embodiment, said neurodegenerative disease is Alzheimer's disease. In one embodiment, said neurodegenerative disease is dementia. In one embodiment, said neurodegenerative disease includes, but is not limited to, Huntington's disease, amyotrophic lateral sclerosis, multiple sclerosis, and/or Parkinson's disease. In one embodiment, the method further comprises the step of improving brain microvascular and endothelial function. In one embodiment, the method further comprises a step of stimulating brain endothelial nitric oxide synthase activity. In one embodiment, the neurodegenerative disease comprises an early stage Alzheimer's disease In one embodiment, the neurodegenerative disease comprises a mild cognitive impairment. In one embodiment, said stimulated brain endothelial nitric oxide synthase activity improves brain microvascular endothelial function.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement.

The term “substitute for” as used herein, refers to the switching the administration of a first compound or drug to a subject for a second compound or drug to the subject.

The term “suspected of having”, as used herein, refers a medical condition or set of medical conditions (e.g., preliminary symptoms) exhibited by a patient that is insufficient to provide a differential diagnosis. Nonetheless, the exhibited condition(s) would justify further testing (e.g., autoantibody testing) to obtain further information on which to base a diagnosis. The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index, and it can be expressed as the ratio LD50/ED50. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering”, as used herein, refers to any method of providing a composition to a patient such that the composition has its intended effect on the patient. An exemplary method of administering is by a direct mechanism such as, local tissue administration (i.e., for example, extravascular placement), oral ingestion, transdermal patch, topical, inhalation, suppository etc.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

### DETAILED DESCRIPTION OF THE INVENTION

The present invention is in the field of neurodegenerative diseases. In particular, a combined treatment regimen including, but not limited to, including simvastatin, L-arginine, and/or tetrahydrobiopterin is identified that improves cognitive function in patients diagnosed with a neurodegenerative disease (e.g., Alzheimer's disease). Such drugs may improve cerebral microvascular endothelial function by introducing progenitor endothelial cells into the cerebral microvascular circulation. Alternatively, the proposed mechanism is consistent with the use of endothelin-1 inhibitors to prevent, arrest or modify the progression of AD and related disorders.

The “amyloid hypothesis” has dominated Alzheimer research for more than 20 years, and proposes that amyloid is the toxic cause of neural/synaptic damage and dementia. If correct, decreasing the formation or removing amyloid should be therapeutic. Despite discrepancies in the proposed mechanism, and failed clinical trials, amyloid continues to be considered the cause of a degenerative cascade. Alternative hypotheses must explain three features, (i) why amyloid toxicity is not the etiology of Alzheimer's disease (AD), (ii) what alternative mechanisms cause the degeneration and dementia of AD, and (iii) why increased amyloid accumulates in the brain in AD. We propose that AD, which occurs in elderly, already vulnerable brains, with multiple age-related changes, is precipitated by impaired microvascular function, resulting primarily from decreased Notch-related angiogenesis. With impaired microvasculature, a lack of vascular endothelial-derived trophic factors and decreased cerebral blood flow cause the atrophy of neural structures. Therapeutic strategies should focus on supporting normal angiogenesis.

In one embodiment, the present invention contemplates compositions and methods for inducing angiogenesis of brain blood vessel angiogenesis. Although it is not necessary to understand the mechanism of an invention, it is believed that one mechanism underlying and precipitating the development of AD may be a failure of trophic function due to impaired cerebral blood vessel angiogenesis. Drachman, D. A., Alzheimer's and Dementia 10(3):372-380 (2014). It is also believed that impaired cerebral blood vessel angiogenesis in patients with AD may be due to a number of mechanisms including, but not limited to, an impaired presenilin-induced Notch cleavage. Such an impaired Notch cleavage would be expected to affect brains of elderly individuals, so that the brains become vulnerable due to multiple age-related changes. Drachman, D. A., Neurology 67:1340-1352 (2006).

In one embodiment, the present invention contemplates a method for improving brain microvascular endothelial function thereby maintaining normal and healthy brain function. In one embodiment, the method contemplates preventing cerebral atrophic changes associated with advancing age. In one embodiment, the method further comprises preventing a cognitive decline. In one embodiment, the method further comprises preventing Alzheimer's dementia.

In one embodiment, the present invention contemplates a method for facilitating cerebral microvessel endothelial cell lining function. Although it is not necessary to understand the mechanism of an invention, it is believed that improved cerebral microvessel endothelia cell lining function improves brain function in patients with Alzheimer's disease and other related degenerative disorders.

### I. Alzheimer's Disease

Alzheimer's Disease (AD) is believed to be a neurodegenerative disorder of unknown etiology, characterized by symptoms including, but not limited to, progressive dementia, postmortem senile plaque pathology and brain neurofibrillary tangles. Although it is not necessary to understand the mechanism of an invention, it is believed that AD may be caused by rare (3%) dominant genetic mutations including, but not limited to, amyloid precursor protein (APP), presenilin 1 and presenilin 2. It is believed that these mutations may occur as an Early Onset Dominantly Inherited (EODI) form or as a Late Onset Sporadic AD (LOSAD). LOSAD is sporadic in appearance but may be associated with more common genetic risk factors including, but not limited to, an apolipoprotein E mutation (i.e., for example, APOEε4). LOSAD is believed to be responsible for approximately 95% of AD patients. Age, diabetes, hypertension and/or dyslipidemia are also risk factors related to AD incidence. While much research has been devoted to the “amyloid hypothesis,” asserting that beta amyloid (Aβ) formation is the underlying cause of AD, it remains uncertain as to whether Aβ is actually a cause of AD, or simply a secondary consequence, thereby serving only as an AD “biomarker”. For example, Aβ is found in brains during normal aging and clinical trials of drugs that block the formation of Aβ, or remove it, have not produced a benefit. Further, lifetime accumulations of brain Aβ have not been shown to relate to the severity of dementia or Alzheimer's disease.

At present, the etiology of LOSAD remains unknown. Iqbal et al., “Stratification of patients is the way to go to develop neuroprotective/disease-modifying drugs for Alzheimer's disease” J Alzheimer's Dis 15:339-345 (2008); Drachman D. A., “Aging of the brain, entropy, and Alzheimer disease” Neurology 67:1340-1352 (2006); Bertram et al., “Thirty years of Alzheimer's disease genetics, the implications of systematic meta-analyses” Nat Rev Neurosci 9:768-778 (2008); and Miller et al., “Do early-life insults contribute to the late-life development of Parkinson and Alzheimer diseases?” Metabolism 57 Suppl 2. S44-49 (2008). Characterized by gradual-onset, progressive dementia, LOSAD's cerebral pathology is usually defined by neuritic plaques containing fragmented neuritic particles and β-amyloid (AP), and by neurofibrillary tangles (NFT). NFTs generally contain paired helical filaments comprised of hyperphosphorylated tau protein. Loss of synapses and neurons in combination with increased cortical atrophy are also associated with a decline in cognitive function. Scheff et al., “Synaptic alterations in CA1 in mild Alzheimer disease and mild cognitive impairment” Neurology 68:1501-1508 (2007); Hamos et al., “Synaptic loss in Alzheimer's disease and other dementias” Neurology 39:355-361 (1989); Lippa et al., “Alzheimer's disease and aging: effects on perforant pathway perikarya and synapses” Neurobiol Aging 13:405-411 (1992); and Rabinovici et al., “Distinct MRI atrophy patterns in autopsy-proven Alzheimer's disease and frontotemporal lobar degeneration” Am J Alzheimer's Dis Other Demen 22:474-488 (2007).

These changes often, but not always, begin in the medial temporal lobe structures, including, but not limited to, the entorhinal cortex and/or the hippocampal areas. LOSAD pathology may be indistinguishable from that seen in Early-Onset Dominantly-Inherited AD (EODI AD) which appears to be caused by mutations of genes related to the formation of AP, as well as to Notch1 activation. Takeshita et al., “Critical role of endothelial Notch1 signaling in postnatal angiogenesis” Circ Res 100:70-78 (2007). Even so, it is not clear whether AP, or its precursor protein (APP), have a primary causal role in the development of neural degeneration and dementia in EODI AD. Furthermore, the relationship between β-amyloid and LOSAD is even less clear.

As one indication of this uncertainty, therapeutic efforts to remove amyloid in humans (e.g., by immunologic means), or to interfere with its production by blockade of the key beta and gamma secretases that cleave Aβ from APP, have so far been ineffective in treating or preventing AD. Williams M., “Progress in Alzheimer's disease drug discovery: An update” Curr Opin Investig Drugs 10:23-34 (2009); and Holmes et al., “Long-term effects of Abeta42 immunisation in Alzheimer's disease, follow-up of a randomised, placebo-controlled phase I trial” Lancet 372:216-223 (2008). Other studies have failed to determine that the amount of Aβ imaged by positron emission tomography (PET) scanning with Pittsburgh Compound B (C-PIB) is clearly related to cognitive impairment. Aizenstein et al., “Frequent amyloid deposition without significant cognitive impairment among the elderly” Arch Neurol 65:1509-1517 (2008). Despite the evidence that Aβ dimers can be toxic to synapses, there is increasing doubt regarding the hypothesized principal role of Aβ as the fundamental etiology of sporadic AD, suggesting that the accumulation of amyloid may be a downstream effect, or a biomarker of an underlying degenerative process. Selkoe D J., “Soluble oligomers of the amyloid beta-protein impair synaptic plasticity and behavior” Behav Brain Res 192:106-113 (2008); and Xu et al., “Mid- and late-life diabetes in relation to the risk of dementia: a population-based twin study” Diabetes 58:71-77 (2009).

Without consensus of a clear mechanism for the origination and/or development of AD, there may be several probable mechanisms worthy of consideration. For example, one LOSAD risk factor LOSAD advancing age. It is generally believed that the risk of developing AD doubles every 5 years after age 65, with almost 50% of individuals showing significant cognitive impairment by age 85. Ziegler-Graham et al., “Worldwide variation in the doubling time of Alzheimer's disease incidence rates” Alzheimer's Dement 4:316-323 (2008). The annual incidence of AD at 85 is 20-30 times greater than at 65. Seshadri et al., “Apolipoprotein E epsilon 4 allele and the lifetime risk of Alzheimer's disease. What physicians know, and what they should know” Arch Neurol 52:1074-1079 (1995). Other identified risk factors include, but are not limited to the presence of an apolipoprotein Eε4 allele, hypertension, diabetes, and/or dyslipidemia. Breteler et al., “Risk factors for vascular disease and dementia” Haemostasis 28:167-173 (1998); Cechetto et al., “Vascular risk factors and Alzheimer's disease” Expert Rev Neurother 8:743-750 (2008); Profenno et al., “Diabetes and overweight associate with non-APOE4 genotype in an Alzheimer's disease population” Am J Med Genet B Neuropsychiatr Genet 1478:822-829 (2008); Reitz et al., “Hypertension and the risk of mild cognitive impairment” Arch Neurol 64:1734-1740 (2007).

In human studies, analyses performed with PET and single-photon emission computed tomography (SPECT) scanning have shown decreased metabolism and cerebral blood flow in patients with AD, particularly in the parietal regions, and involving frontal and temporal areas as well. Herholz et al., “Positron emission tomography imaging in dementia” Br J Radiol 80 Spec No 2:8160-167 (2007); and O'Brien J. T., “Role of imaging techniques in the diagnosis of dementia” Br J Radiol 80 Spec No 2:S71-77 (2007). Microscopic examination of post-mortem AD brain with specific immunohistopathologic stains (CD 31) for microvascular endothelium reveals narrowed, tortuous microvessels with scanty, poorly-stained endothelium in affected AD cortical tissue. While the possibility exists that these vascular changes may be a consequence of altered brain structure, or of diminished functional demand, the consistent epidemiologic association of multiple vascular risk factors with the incidence of AD suggests that the vascular changes are a plausible causal factor, initiating or contributing to the pathogenesis and development of AD.

One line of reasoning suggests that the failure of brain microvasculature may play a role in the pathogenesis of AD. For example, a number of studies support the concept that the microvascular endothelium in brain (and elsewhere) is a major paracrine tissue, secreting trophic factors that are believed to maintain organ/tissue integrity. Santhanam et al., “Endothelial progenitor cells stimulate cerebrovascular production of prostacyclin by paracrine activation of cyclooxygenase-2” Circ Res 100:1379-1388 (2007), Rubio et al., “Nitric oxide, an iceberg in cardiovascular physiology: far beyond vessel tone control” Arch Med Res 35:1-11 (2004); Scumpia et al., “Endothelial heat shock response in cerebral ischemia” Histol Histopathol 22:815-823 (2007); and Shen et al., “Endothelial cells stimulate self-renewal and expand neurogenesis of neural stem cells” Science 304:1338-1340 (2004). This concept, was initially proposed as a potential strategy for cancer therapy, in which tumor tissue deprived of adequate vasculature has been shown to shrink. Folkman J., “Angiogenesis and its inhibitors” Important Adv Oncol 42-62 (1985).

Angiostatin agents, which are believed to block angiogenesis and the integrity and function of vascular endothelium, have been used to control the growth of a number of experimental and human cancers. It has been noted, however, that in experimental animals, transplanted normal tissue will not survive and grow when vascular function is blocked by angiostatin agents, and will undergo atrophy. Folkman J., “Angiogenesis and its inhibitors” Important Adv Oncol 42-62 (1985). Whether this is due to inadequate blood flow (the conduit function of blood vessels), or to the paracrine functions of endothelium, is not known. Factors secreted by endothelial cells in cell culture have been shown, for example, to be involved in the survival, growth and differentiation of neural stem cells, emphasizing that these trophic influences play a role in the maintenance and growth of normal neural tissue.

### II. Ineffectiveness Of β-Amyloid Directed Alzheimer's Treatment

### A. Introduction

For more than 20 years, the “amyloid hypothesis” of Alzheimer's disease (AD) has been the leading scientific explanation for this degenerative disorder. Hardy et al., “Alzheimer's disease: the amyloid cascade hypothesis” Science 256:184-185 (1992); and Constantinidis J., “Hypothesis regarding amyloid and zinc in the pathogenesis of Alzheimer disease: potential for preventive intervention” Alzheimer Dis Assoc Disord 5:31-35 (1991). This hypothesis proposes that excess toxic accumulation of β-amyloid (AP) in one or more forms, including but not limited to, compact plaques, diffuse plaques, soluble oligomers, fibrils, and/or protofibrils is the specific cause of AD. Spires et al., “Neuronal structure is altered by amyloid plaques” Rev Neurosci 15:267-278 (2004; Selkoe D. J., “Soluble oligomers of the amyloid beta-protein impair synaptic plasticity and behavior” Behav Brain Res 192.106-113 (2008), and Haass et al., “Soluble protein oligomers in neurodegeneration: lessons from the Alzheimer's amyloid beta-peptide” Nat Rev Mol Cell Biol 8:101-112 (2007). The hallmark neuropathological changes, for example, neuronal and synaptic losses and/or cognitive impairment are considered to result from amyloid-related damage. Evidence for this hypothesis includes, but is not limited to, the presence of amyloid in neuritic plaques in AD, the genetics of dominantly inherited familial AD, involving mutations of amyloid precursor protein (APP) and presenilin (PS) genes; the occurrence of Alzheimer-like changes in middle-age patients with Down syndrome, the molecular biology of Aβ production from APP; the neurotoxicity of amyloid in tissue culture; positron emission tomographic (PET) imaging of amyloid markers in the brain of patients with AD, and observations on transgenic mouse models of AD with human mutant genes.

During the past two decades, more than 18,000 articles on the association of Aβ and AD have been published, and most current therapeutic trials designed to modify disease in AD attempt to prevent the production and accumulation of Aβ in the brain. Scientific and clinical information makes it clear that Aβ is associated with AD. One question, however, relates to causality: Is Aβ the primary cause of late-onset sporadic Alzheimer's disease (LOSAD)? If so, modifying the production, accumulation, circulation, fixation, or removal of Aβ should be the most appropriate strategies for preventing and/or treating AD. Selkoe D. J., “The therapeutics of Alzheimer's disease: where we stand and where we are heading” Ann Neurol 74:328-336 (2013). If Aβ is an epiphenomenon associated with the process or processes that cause late-onset sporadic dementia—or a minor contributing factor to LOSAD-therapeutic efforts should be directed at other targets. The evidence for and against the amyloid hypothesis should be evaluated and alternative explanations considered. Current treatments for AD are of modest, symptomatic benefit, disease-modifying therapies will depend on an accurate understanding of the molecular mechanisms that cause AD.

### B. The β-Amyloid Hypothesis

In 1907, Alois Alzheimer described the clinical and pathological features of a single patient whose dementia started at age 51. Bick K. L., “The early story of Alzheimer disease” In: Terry R D, Katzman R, Bick K L, Sisodia S S, eds. Alzheimer disease. 2nd ed. Philadelphia: Lippincott Williams & Wilkins; p. 1-9 (1999). In 1910, Emil Kraepelin named this presenile dementia “Alzheimer's disease” after his junior associate in his authoritative psychiatric textbook. Schwab et al., “Transgenic mice overexpressing amyloid beta protein are an incomplete model of Alzheimer disease” Exp Neurol 188:52-64 (2004). Neuritic plaques had been described 15 years previously, and neurofibrillary tangles earlier in 1907, but the “Alzheimer's disease” eponym has prevailed. Duff et al., “Transgenic mouse models of Alzheimer's disease: how useful have they been for therapeutic development?” Brief Fund Genomic Proteomic 3:47-59 (2004); Xia et al., “γ-Secretase modulator in Alzheimer's disease: shifting the end” J Alzheimer's Dis 31:685-696 (2012); Berrios G. E., “The history of Alzheimer's disease” Wellcome collection.org. (2004).

For 70 years, AD was considered to be presenile dementia, and was considered very rare. In 1976, Robert Katzman's seminal editorial in the Archives of Neurology stated that presenile and senile dementia were sufficiently similar clinically and neuropathologically to be considered a single condition, identified as AD; this designation has been accepted universally. Katzman R., “The prevalence and malignancy of Alzheimer disease, a major killer [editorial]” Arch Neurol 33:217-218 (1976). The presence of amyloid in the brain of patients with AD had been known since at least the 1920s, particularly as “congophilic angiopathy” in the cerebral and meningeal blood vessels. Divry P., “Etude histo-clinique des plaques seniles” J Belg Neurol Psychiat 27:643-657 (1927). Until the 1980s, however, the role of amyloid was generally considered to be a secondary product of altered immunoglobulins. Glenner G. G., “Current concepts of the formation and composition of amyloid” Ann Clin Lab Sci 5:257-263 (1975). In 1984, Glenner and Wong found that the molecular composition of amyloid from patients with AD was distinctive, and proposed that assessment of Aβ had potential value for diagnostic testing and might be related etiologically to AD. Glenner et al., “Alzheimer's disease: initial report of the purification and characterization of a novel cerebrovascular amyloid protein” Biochem Biophys Res Commun 120:885-890 (1984). In 1991, Alison Goate found that patients in six families with early-onset dominantly inherited AD (EODI AD) had a mutation involving a gene on chromosome 21, which was later shown to code for the APP—a large protein from which the smaller amyloid peptides (AP40 and Aβ42) found in neuritic plaques in AD were derived. Goate et al., “Segregation of a missense mutation in the amyloid precursor protein gene with familial Alzheimer's disease” Nature 349:704-706 (1991), and Chartier-Harlin et al., “Early-onset Alzheimer's disease caused by mutations at codon 717 of the beta-amyloid precursor protein gene” Nature 353:844-846 (1991). During the early 1990s, other patients with EODI AD, but lacking an APP mutation, were found to have different mutations on chromosome 14 or chromosome 1, causing a similar early onset form of familial AD (FAD). Sherrington et al., “Cloning of a gene bearing missense mutations in early-onset familial Alzheimer's disease” Nature 375:754-760 (1995); and St George-Hyslop et al., “Genetic evidence for a novel familial Alzheimer's disease locus on chromosome 14” Nat Genet 2:330-334 (1992). These mutations altered the structure of an enzyme, subsequently named “presenilin,” shown to be part of the γ-secretase complex. γ-Secretase cleaves Aβ42 from the APP protein after an initial cleavage by β-secretase. More than 180 mutations in the PS gene have been found to cause EODI AD, with essentially complete penetrance, and were presumed to increase the production of Aβ42. Hardy et al., “The amyloid hypothesis of Alzheimer's disease: progress and problems on the road to therapeutics” Science 297:353-356 (2002); and Areza-Fegyveres et al., “Dementia pugilistica with clinical features of Alzheimer's disease” Arq Neuropsiquiatr 65:830-833 (2007).

During the 1990s, a transgenic mouse model of FAD was engineered using the FAD mutant APP human gene, which resulted in transgenic (TG) mice with amyloid-containing plaques, slight behavioral changes late in life, but little neuronal or synaptic loss in the hippocampus. Hsiao et al., “Correlative memory deficits, Abeta elevation, and amyloid plaques in transgenic mice” Science 274:99-102 (1996); and West et al., “Synaptic contact number and size in stratum radiatum CA1 of APP/PS1 DeltaE9 transgenic mice” Neurobiol Aging 30:1756-76 (2009). This evidence focused attention on amyloid (Aβ42) in the brain of patients with AD. Amyloid-related genetic mutations in the rare FAD suggested that amyloid in the brain might cause AD—not only in EODI FAD, but also in LOSAD as well. Clinical research with amyloid-binding radioisotope ligands (i.e., for example, Pittsburgh compound B, florbetapir), which can be imaged with PET scanning, has shown increased amyloid in the brain of patients with AD, providing additional support for this concept. Klunk et al., “Imaging brain amyloid in Alzheimer's disease with Pittsburgh compound-B” Ann Neurol 55:306-319 (2004); and Fleisher et al., “Using positron emission tomography and florbetapir F18 to image cortical amyloid in patients with mild cognitive impairment or dementia due to Alzheimer disease” Arch Neurol 68:1404-1411 (2011).

Pharmaceutical companies and investigators have developed drugs and immunological agents to reduce the production of Aβ42 or to remove fixed amyloid in neuritic plaques. To date, a number of clinical trials have been completed; none have improved cognitive function, although several have effectively removed Aβ42. Rinne et al., “11C-PiB PET assessment of change in fibrillar amyloid-beta load in patients with Alzheimer's disease treated with bapineuzumab: a phase 2, double-blind, placebo-controlled, ascending-dose study” Lancet Neurol 9:363-372 (2010); and Nicoll et al., “Neuropathology of human Alzheimer disease after immunization with amyloid-beta peptide, a case report” Nat Med 9:448-452 (2003). Questions regarding the logic implicating Aβ as the specific, primary cause of LOSAD, and the failure so far to derive clinical benefit from removing brain amyloid in patients with AD, are highly relevant to determining the future of clinically effective treatments for AD.

### C. Critical Assessment Of β-Amyloid Alzheimer's Disease Evidence

Many published articles address the relationship between amyloid and AD. The following is a critical assessment of research that supports amyloid as being a cause of AD:

1) The genetic abnormalities in EODI FAD and Down syndrome, involving amyloid-related mechanisms, provide the most compelling support for amyloid as the cause of LOSAD. Tanzi et al., “Twenty years of the Alzheimer's disease amyloid hypothesis: a genetic perspective” Cell 120:545-555 (2005). Whether sporadic AD is a result of the same mechanism as EODI FAD, and whether the genetic abnormalities indicate that amyloid is the cause of the pathology and dementia in FAD, remain unproved.

2) By definition, LOSAD has a different etiology from EODI FAD and Down syndrome, lacking the genetic abnormalities that produce those conditions. It was presumed initially that in LOSAD, as in the other conditions, the amount of Aβ in brain was increased, and that toxicity of Aβ damaged proximate neurons.

3) It is now clear that amyloid plaques are not adjacent to neurons or synapses lost early in AD. Terry et al., “The neuropathology of Alzheimer disease and the structural basis of its cognitive alterations” In; Terry et al., eds. Alzheimer disease. 2nd ed. Philadelphia. Lippincott Williams & Wilkins; p. 187-206 (1999). Neurons are typically lost initially in the hippocampus and entorhinal cortex, whereas amyloid plaques are first found in frontal regions, basal ganglia, or elsewhere. How distant plaques might damage neurons or synapses remains unclear.

4) The amount of amyloid in AD brain is not related directly to the extent of cognitive decline. Gomez-Isla et al., “Neuronal loss correlates with but exceeds neurofibrillary tangles in Alzheimer's disease” Ann Neurol 41:17-24 (1997); Drachman D. A., “Case records of the Massachusetts General Hospital: weekly clinicopathological exercises, case 12-1999: a 67-year-old man with three years of dementia” N Engl J Med 340:1269-1277 (1999); and Tokuda et al., “Re-examination of ex-boxers' brains using immunohistochemistry with antibodies to amyloid beta-protein and tau protein” Acta Neuropathol 82:280-285 (1991). The absence of an amyloid-related “dose effect” for the amount of neuronal loss and degree of dementia raises questions regarding the toxic effect of amyloid on the brain.

5) Many cognitively normal elderly subjects have relatively large amounts of Ab in their brain postmortem. Price et al., “Neuropathology of nondemented aging: presumptive evidence for preclinical Alzheimer disease” Neurobiol Aging 30:1026-1236 (2009); Davis et al., “Alzheimer neuropathologic alterations in aged cognitively normal subjects” J Neuropathol Exp Neurol 58:376-388 (1999), Balasubramanian et al., “Alzheimer disease pathology and longitudinal cognitive performance in the oldest-old with no dementia” Neurology 79:915-921 (2012). Recently, imaging Aβ with Pittsburgh compound B and florbetapir, PET studies in cognitively normal subjects showed that almost a third of elderly individuals have major amounts of Aβ in their brain. Rowe et al., “Imaging beta-amyloid burden in aging and dementia” Neurology 68:1718-1725 (2007); and Rodriguez et al., β-Amyloid burden in healthy aging: regional distribution and cognitive consequences” Neurology 78:387-395 (2012). This suggests that cerebral Aβ plaques are not sufficient, to cause AD. Suggestions that cognitively normal elderly subjects with neuritic plaques have “preclinical AD” cannot be evaluated postmortem. Although Aβ neuritic plaques are associated with AD, they may serve as a marker of the process that can result in neuronal loss and eventually dementia, but do not demonstrate causality.

6 Neuropathological criteria emphasize that fewer neuritic plaques are required to diagnose AD in the brain of younger people than older people, contrary to the expected greater vulnerability of older subjects' brains. Khachaturian Z. S., “Diagnosis of Alzheimer's disease” Arch Neurol 42:1097-1105 (1985). This discrepancy suggests that Aβ plaque formation is not necessarily the cause of neuronal loss and dementia, but an unrelated marker of the degenerative process.

7) Very elderly subjects in their 90s and 100s can develop dementia similar to that in younger patients with LOSAD, but with slower evolution. At autopsy, they frequently have few neuritic plaques and may lack other pathological features of AD, indicating that amyloid plaques are not necessary for the clinical syndrome of AD. The loss of neurons and synapses can thus be independent of amyloid. Middleton et al., “Neuropathologic features associated with Alzheimer disease diagnosis, age matters” Neurology 77:1737-1744 (2011); Reitz C., “Alzheimer's disease and the amyloid cascade hypothesis: a critical review” Int J Alzheimer's Dis 2012:369808 (2012); and Imhof et al., “Morphological substrates of cognitive decline in nonagenarians and centenarians: a new paradigm?”. J Neurol Sci 257:72-79 (2007).

8) The mechanism of AD was initially thought to be a toxic gain-of-function of Aβ production in FAD, and age-/time-related increased accumulation of Aβ in elderly subjects with LOSAD. It is now clear that most PS mutations produce a loss of function, with decreased amounts of total Aβ production. Geda Y. E., “Mild cognitive impairment in older adults” Curr Psychiatry Rep 14:320-327 (2012); and Van Broeck et al., “Current insights into molecular mechanisms of Alzheimer disease and their implications for therapeutic approaches” Neurodegener Dis 4:349-365 (2007). Only the ratio of Aβ42 to Aβ40 production is increased in patients with FAD; the longer Aβ42 fragments are said to be more toxic. Wolfe M S., “When loss is gain: reduced presenilin proteolytic function leads to increased Abeta42/Abeta40: talking point on the role of presenilin mutations in Alzheimer disease” EMBO Rep 8:136-140 (2007).

9) When dementia, and neuronal/synaptic loss failed to correlate with the number of neuritic plaques, investigators proposed that soluble amyloid oligomers are the toxic form of Aβ damaging neurons and, particularly, synapses (supra). Although amyloid oligomers are not visualized either in vivo or postmortem, tissue culture studies show they can interfere with postsynaptic potentiation in vitro. Petersen R. C., “Mild cognitive impairment: current research and clinical implications” Semin Neurol 27:22-31 (2007). The amount of amyloid oligomers needed to produce this effect is typically larger than physiological amounts in the brain, nor are fixed neuritic plaques likely to be a “reservoir” from which soluble circulating oligomers are eluted, because of the tenacity of plaques for polymerization and fixation of Ab fragments. Esler et al., “Alzheimer's disease amyloid propagation by a template-dependent dock-lock mechanism” Biochemistry 39:6288-6295 (2000).

10) Transgenic mouse models of FAD provide limited support for Aβ toxicity causing neuronal damage in AD. Schaeffer et al., “Insights into Alzheimer disease pathogenesis from studies in transgenic animal models” Clinics (Sao Paulo) 66:45-54 (2011). To produce significant neuropathological change in TG mice, double transgenic mice—with both the APP and PS mutations—were developed. Although they develop neuritic plaques with aging, decline of cognitive (behavioral) function is limited, and neuropathology reveals minimal neuronal or synaptic loss and few neurofibrillary tangles. Irizarry et al., “APPSw transgenic mice develop age-related A beta deposits and neuropil abnormalities, but no neuronal loss in CA1” J Neuropathol Exp Neurol 56:965-973 (1997). TG mice are a questionable model of LOS AD.

11) A most troubling observation is the repeated clinical failure of agents that interfere with Aβ production, or reduce the Aβ burden, in human trials of AD. Various therapeutic strategies have removed the amyloid in plaques with active or passive immunotherapy, interfered with amyloid oligomer formation, disrupted amyloid transport via receptor for advanced glycation endproducts inhibition, blocked or modified γ-secretase; or broken Aβ peptides. These drugs, however, have neither arrested progression of cognitive decline nor improved memory. Especially notable was the failure of the AN-1792 (Elan Pharmaceuticals, Inc.) active immunization trial, stopped when several subjects developed an autoimmune encephalopathy. Gilman et al., “Clinical effects of Abeta immunization (AN1792) in patients with AD in an interrupted trial” Neurology 64:1553-1562 (2005). Active immunization decreased Aβ markedly in the brain of treated subjects, but failed to decrease the progression of dementia, even when the amyloid plaques had been removed. Serrano-Pozo et al., “Beneficial effect of human anti-amyloid-beta active immunization on neurite morphology and tau pathology” Brain 133.1312-1327 (2010); Tabira T., “Immunization therapy for Alzheimer disease: a comprehensive review of active immunization strategies” Tohoku J Exp Med 220:95-106 (2010), Holmes et al., “Long-term effects of Abeta42 immunisation in Alzheimer's disease: follow-up of a randomised, placebo-controlled phase I trial” Lancet 372:216-223 (2008). Quantitative imaging studies showed greater brain atrophy in the brain of treated subjects with immunological response and decreased amyloid burden than in control subjects. Fox et al., “Effects of Abeta immunization (AN1792) on MRI measures of cerebral volume in Alzheimer disease” Neurology 64:1563-1572 (2005).

12) Other attempts to decrease production of Aβ or reduce fibrillization have also failed to improve cognition or arrest decline. Aisen et al., “Tramiprosate in mild-to-moderate Alzheimer's disease: a randomized, double-blind, placebo-controlled, multi-centre study (the Alphase Study)” Arch Med Sci 7:102-111 (2011); Kerchner et al., “Bapineuzumab” Expert Opin Biol Ther 10:1121-1130 (2010); Salomone et al., “New pharmacological strategies for treatment of Alzheimer's disease: focus on disease modifying drugs” Br J Clin Pharmacol 73:504-517 (2012), Herrmann et al., “Current and emerging drug treatment options for Alzheimer's disease, a systematic review” Drugs 71:2031-2065 (2011). The failure of semagacestat—an agent inhibiting PS—was striking. Panza et al., “Interacting with γ-secretase for treating Alzheimer's disease, from inhibition to modulation” Curr Med Chem 18:5430-5447 (2011); Imbimbo et al., “γ-Secretase inhibitors and modulators for the treatment of Alzheimer's disease: disappointments and hopes” Curr Top Med Chem 11:1555-1570 (2011); and Doody et al., “A phase 3 trial of semagacestat for treatment of Alzheimer's disease” N Engl J Med 369:341-350 (2013). This trial was stopped prematurely when treated subjects showed significantly worse cognitive performance than control subjects—a change attributed to semagacestat's interference with the ability of g-secretase to cleave Notch1 (discussed later). Avagacestat, a similar drug inhibiting γ-secretase cleavage of APP, but with less effect on Notch, has also produced no cognitive benefit in a phase 2 study. Tarenflurbil, a γ-secretase modulator, also failed to produce any benefit. Xia et al., “γ-Secretase modulator in Alzheimer's disease, shifting the end” J Alzheimer's Dis 31.685-696(2012).

### D. β-Amyloid is an Alzheimer's Biomarker—not a Cause

Together, these observations suggest that the accumulation of Aβ and the development of neuritic plaques, are neither necessary nor sufficient to produce the neuronal losses and clinical changes of AD. Although the neuropathological appearance of Aβ is associated with the development of AD, evidence for causality is inconsistent. Neuropathological changes, neuronal and synaptic losses, and eventually dementia occur in AD, but Aβ is not related quantitatively to the losses, not anatomically proximate to the changes, and not a direct consequence of the aging process; and the neuropathological and clinical damage has not been prevented or arrested by pharmacological or immunological removal of fixed or circulating Aβ. Biomarkers of AD, including Aβ accumulation, precede dementia by many years, but the idea that deposition of amyloid triggers a cascade of undefined neurodegenerative events is yet to be supported by specific evidence, den Heijer et al., “Vascular risk factors, apolipoprotein E, and hippocampal decline on magnetic resonance imaging over a 10-year follow-up” Alzheimer's Dement 8.417-425 (2012). After more than 20 years of intense investigation on the relation of Aβ and AD, with tens of millions of dollars spent to develop disease-modifying treatments for AD based on amyloid reduction, the burden of evidence now indicates that amyloid toxicity in the brain resulting from a gain of function is increasingly unlikely to be the cause of sporadic AD.

### E. Questioning the Amyloid Hypothesis

A number of reports have increasingly questioned the relation of Aβ to dementia in AD. As discussed above, Terry et al. cast doubt on a causal role of amyloid in AD on neuropathological grounds. Another report found that the association of neurofibrillary tangles was more clearly relevant to the dementia of AD than the presence of amyloid-containing neuritic plaques. Braak et al., “Neuropathological staging of Alzheimer-related changes” Acta Neuropathol 82:239-259 (1991). Previously, it was noted that aging of the brain, and multiple entropic changes, are major contributors to dementia of the elderly. Drachman D. A., “Aging of the brain, entropy, and Alzheimer disease” Neurology 67:1340-1352 (2006). Others have raised additional questions about the validity of the amyloid hypothesis of AD. Pimplikar S. W. “Reassessing the amyloid cascade hypothesis of Alzheimer's disease” Int J Biochem Cell Biol 41:1261-1268 (2009); Pimplikar et al., “Amyloid-independent mechanisms in Alzheimer's disease pathogenesis” J Neurosci 30:14946-4954 (2010); Herrup K., “Reimagining Alzheimer's disease: an age-based hypothesis” J Neurosci 30:16755-16662 (2010); Fjell et al., “Neuroimaging results impose new views on Alzheimer's disease: the role of amyloid revised” Mol Neurobiol 45:153-172 (2012); and Armstrong R. A., “The pathogenesis of Alzheimer's disease: a reevaluation of the “amyloid cascade hypothesis” Int J Alzheimer's Dis 2011:630865 (2011).

If amyloid is not the cause of sporadic Alzheimer's dementia, it is necessary to consider (i) what other mechanisms might be the etiologies of sporadic AD and (ii) why amyloid is a frequent-if not universal-accompaniment of AD, if it is not the cause.

### F. Alternative Explanations

LOSAD dementia is not the result of a single cause, but results from the confluence of “soil” and “seed”-multiple age-associated processes that erode brain structure and function gradually, making it vulnerable to degeneration—combined with precipitating conditions that trigger the events of AD, resulting in accelerated neuronal and synaptic losses, and cognitive decline. Masoro E. J., “Are age-associated diseases an integral part of aging? In; Masoro E J, Austad S N, eds. Handbook of the biology of aging. 6^{th} ed. Burlington, Mass. Elsevier Academic Press, 2006. p. 43-62.

1. Soil

Normal age-related changes Decline of cognition and loss of neurons and their connections are well-documented accompaniments of normal aging. Salthouse T. A., “Selective review of cognitive aging” J Int Neuropsychol Soc 16:754-760 (2010). Cognitive decline with age—recognized for centuries—has been studied intensively by psychologists and designated as “age-associated memory impairment” or “mild cognitive impairment” Salthouse T. A., “Adult cognition” New York: Springer-Verlag; (1982); and Hoyer et al., “Memory aging” In; Birren J E I, Schaie K W, eds. Handbook of the psychology of aging. 6th ed. Burlington, Mass.:Elsevier Academic Press, 2006. p. 209-224. Nonagenarians and centenarians almost invariably experience significant cognitive impairment, without the accelerated decline of AD or the accumulation of neuritic plaques and neurofibrillary tangles. The loss of brain weight, volume, neurons, and connections has been well documented. In the absence of AD, unbiased stereological techniques show that by age 90, nearly 10% of neocortical neurons, and about 40% of axonal and dendritic connections are lost. Pakkenberg et al., “Aging and the human neocortex” Exp Gerontol 38:95-99 (2003); and Marner et al., “Marked loss of myelinated nerve fibers in the human brain with age.” J Comp Neurol 462:144-152 (2003). Numerous changes similar to those affecting the heart, lungs, muscles, and liver may cause neural and synaptic losses. Intrinsic factors affecting brain longevity include, but are not limited to: i) differing initial endowment and/or replicative senescence of supporting tissues, collectively known as the Hayflick phenomenon (Hayflick L., “Intracellular determinants of cell aging” Mech Ageing Dev 28:177-185 (1984): and Hayflick L., “The cell biology of aging” Clin Geriatr Med 1:15-27 (1985); ii) telomeric shortening (Rodriguez-Brenes et al., “Quantitative theory of telomere length regulation and cellular senescence” Proc Natl Acad Sci USA 107:5387-5392 (2010); and iii) apoptotic losses, and aging of replacement stem cells (Conover et al., “Aging of the subventricular zone neural stem cell niche” Aging Dis 2:49-63 (2011); and Liu et al., “Aging of stem cells, intrinsic changes and environmental influences” In; Masoro E J, Austad S N, eds. Handbook of the biology of aging. 7th ed. Amsterdam: Elsevier; 2011. p. 141-61. Extrinsic factors leading to decline of brain structure may include, but are not limited to, molecular misreading, oxidative stress, mitochondrial damage, advanced glycation end products, conformational alteration of proteins, and/or microvascular endothelial losses. Lustgarten et al., “An objective appraisal of the free radical theory of aging” In: Masoro E J, Austad S N, eds. Handbook of the biology of aging. 7th ed. Amsterdam: Elsevier; 2011. p. 177-202.

The combination of intrinsic and stochastic events typically contributes to gradual, and inevitable, physical decline of the brain during the passage of time. Despite this, some elderly individuals retain cognitive integrity to advanced old age whereas others undergo losses. The vulnerable aged brain, with normal age-associated changes, may be affected by an additional precipitating event that transforms it from normal aging to AD.

2. “Seed”

Precipitating events causing AD Increasing evidence casts doubt on toxicity by Aβ, resulting from a genetic or acquired gain of function, as a cause of sporadic AD. Absent Aβ toxicity as an etiology, a number of clues to the etiology of AD may lead in another way. For example, one explanation of a mechanism for AD should include, but is not limited to, a role of aging, association with vascular risk factors, a relation to PS mutations, an accumulation of amyloid, early development of AD pathology in Down syndrome, an early occurrence of cerebral atrophy, a consistency of hippocampal involvement, and perhaps an inverse relation between cancer and AD.

A mechanism encompassing these features-however complex—may cast light on this elusive puzzle. Vascular risk factors have long been associated with degenerative dementia, de la Torre J. C., “The vascular hypothesis of Alzheimer's disease: bench to bedside and beyond” Neurodegener Dis 7.116-121 (2010), and Marchesi V. T., “Alzheimer's dementia begins as a disease of small blood vessels, damaged by oxidative-induced inflammation and dysregulated amyloid metabolism, implications for early detection and therapy” FASEB J 25:5-13 (2011). This association is distinct from vascular dementia, which (although still evolving) is determined by the volume of brain destruction, location of vascular lesions, number of cerebrovascular lesions, and exacerbation of preexisting AD by new infarctions. Jellinger K. A., “Pathology and pathogenesis of vascular cognitive impairment: a critical update” Frontiers Aging Neurosci 5:17 (2013); and Roman et al., “Vascular dementia: diagnostic criteria for research studies, report of the NINDS-AIREN international workshop” Neurology 43:250-260 (1993). The Rotterdam study found that subjects who smoked, had diabetes, or had atherosclerosis had an increased risk of developing AD. Ott et al., “Association of diabetes mellitus and dementia: the Rotterdam study” Diabetologia 39:1392-1397 (1996); Hofman et al., “Atherosclerosis, apolipoprotein E, and prevalence of dementia and Alzheimer's disease in the Rotterdam study” Lancet 349:151-154 (1997); and Breteler M. M., “Vascular risk factors for Alzheimer's disease: an epidemiologic perspective” Neurobiol Aging 21.153-160 (2000). People with untreated diastolic hypertension or white matter microvascular hyperintensities on magnetic resonance images showed increased hippocampal atrophy during 10 years of follow-up. Midlife hypertension, diabetes, smoking, and obesity were associated with late life AD, global and hippocampal cerebral atrophy, and decreased executive function later in life in studies in Goteborg and in Framingham. Debette et al., “Midlife vascular risk factor exposure accelerates structural brain aging and cognitive decline” Neurology 77:461-468 (2011); and Skoog et al., “Update on hypertension and Alzheimer's disease” Neurol Res 28:605-611 (2006). Others have demonstrated decreased microvascular density in the cerebral cortex of patients with AD, and numerous atrophic microvascular “string” vessels. Buee et al., “Pathological alterations of the cerebral microvasculature in Alzheimer's disease and related dementing disorders” Acta Neuropathol 87:469-480 (1994); Buee et al., “Brain microvascular changes in Alzheimer's disease and other dementias” Ann N Y AcadSci 826:7-824 (1997); and Brown et al., “Review: cerebral microvascular pathology in ageing and neurodegeneration” Neuropathol Appl Neurohiol 37:56-74(2011).

Regional cerebral blood flow has been reported to be diminished in AD. Austin et al., “Effects of hypoperfusion in Alzheimer's disease” J Alzheimer's Dis 26:123-133 (2011); and Mazza et al., “Primary cerebral blood flow deficiency and Alzheimer's disease, shadows and lights” J Alzheimer's Dis 23:375-389 (2011). Normal cerebral microvasculature preserves integrity of the brain—in part by maintaining cerebral blood flow and in part as a result of the paracrine function of the microvascular endothelium, known to secrete trophic factors critical to maintain organ/tissue integrity. Shen et al., “Endothelial cells stimulate self-renewal and expand neurogenesis of neural stem cells” Science 304:1338-1340 (2004); Li et al., “The impact of paracrine signaling in brain microvascular endothelial cells on the survival of neurons” Brain Res 1287:28-38 (2009). Functioning endothelial cells are, in addition, neuroprotective. Guo et al., “Neuroprotection via matrix-trophic coupling between cerebral endothelial cells and neurons” Proc Natl Acad Sci USA 105:7582-7587 (2008). Widespread microvascular attrition in patients with diabetes, hypertension, and atherosclerosis is capable of contributing to cerebral atrophy and cognitive dysfunction.

Can this vascular impairment produce, in addition, the “hallmark” Aβ accumulation seen postmortem and in imaging during life, with Aβ-containing neuritic plaques? At least two mechanisms explain the accumulation of Aβ in association with microvascular impairment. First, the cerebral microvasculature undergoes remodeling during aging, often with resulting diminution of microcirculation. Faraci F. M., “Cerebral vascular dysfunction with aging” In: Masoro E J, Austad S N, eds. Handbook of the biology of aging. Burlington, Mass. Academic Press Elsevier; 2011. p. 405-19. Angiogenesis is relevant for AD development, it depends in part on cleavage of Notch-1 by PS, in competition with APP. For example, after ligand binding with jagged and 5-like ligands. Notch is cleaved by PS, producing the active Notch intracellular domain, which (interacting with vascular endothelial growth factor) guides the process of angiogenesis by directing microvessel “tip” and “stalk” differentiation. Because both Notch-1 and APP (as well as other proteins) depend on PS for cleavage to their active intracellular domain states, they have been shown to compete for cleavage by PS, based on the amount of substrate present. Berezovska et al., “Notch1 and amyloid precursor protein are competitive substrates for presenilin1-dependent γ-secretase cleavage” J Biol Chem 276:30018-30023 (2001). When less Notch is cleaved, more APP is cleaved, and vice versa. Decreased Notch cleavage impairs angiogenesis, and because normal microvascular function is necessary to maintain the integrity of the brain-both for maintenance of blood flow and for trophic function—the brain shrinks, with loss of synapses, axons, and neurons. Bailey et al., “The nature and effects of cortical microvascular pathology in aging and Alzheimer's disease” Neurol Res 26:573-578 (2004). Decreased Notch cleavage may also contribute to memory impairment by an additional mechanism; in experimental models, reduced active Notch decreases memory. Presente et al., “Notch is required for long-term memory in Drosophila” Proc Natl Acad Sci USA 101:1764-1768 (2004); and Wang et al., “Involvement of Notch signaling in hippocampal synaptic plasticity” Proc Natl Acad Sci USA 101:9458-9462 (2004). The importance of decreased Notch cleavage and impaired microvascular function in AD, rather than Aβ toxicity, is supported by other observations, although requiring considerable additional demonstration, for example:

i) Mutations of PS (PS1, PS2) that cause AD not only reduce APP cleavage by γ-secretase, but also decrease markedly cleavage of Notch-1 by PS. Song et al., “Proteolytic release and nuclear translocation of Notch-1 are induced by presenilin-1 and impaired by pathogenic presenilin-1 mutations” Proc Natl Acad Sci USA 96:6959-6963 (1999).

ii) Notch is highly concentrated in the hippocampus and is important in maintaining neural stem cell function. Berezovska et al., “Notch is expressed in adult brain, is coexpressed with presenilin-1, and is altered in Alzheimer disease” J Neuropathol Exp Neurol 57:738-745 (1998); and Hitoshi et al., “Notch pathway molecules are essential for the maintenance, but not the generation, of mammalian neural stem cells” Genes Dev 16:846-858 (2002).

iii) Semagacestat, the g-secretase modulator used to treat AD in experimental human trials, decreases not only APP cleavage, but also Notch cleavage and function. Semagacestat exacerbated cognitive decline compared with placebo. Although drug failure was attributed to complicating negative effects on Notch function, this supports the concept that decreased Notch function may cause the dementia of AD.

iv) Notch 1 cleavage by PS declines in the brain with older age. Sagare et al., “Neurovascular defects and faulty amyloid-beta vascular clearance in Alzheimer's disease” J Alzheimer's Dis 33Suppl 1:S87-100 (2012). As a consequence, angiogenesis, and the trophic influence of normal microvascular function on integrity of the brain, may decline. As Notch cleavage declines, PS becomes available to cleave more APP, producing increased cerebral Ab in the elderly—a marker of the underlying problem.

v) It has been reported that conditional deletion of Notch genes did not cause excitatory neurons to undergo degeneration. How this relates to Notch-dependent angiogenesis, and endothelial trophic influence on neural survival, is not entirely clear, however. Zheng et al., “Conditional deletion of Notch1 and Notch2 genes in excitatory neurons of postnatal forebrain does not cause neurodegeneration or reduction of Notch mRNAs and proteins” J Biol Chem 287:20356-20368 (2012).

Aβ is believed to be secreted constitutively in the brain, and has a normal function, including, but not limited to: i) increased neurogenesis (Lopez-Toledano et al., “Neurogenic effect of beta-amyloid peptide in the development of neural stem cells” J Neurosci 24:5439-5444 (2004)); ii) protection against stress by free oxygen radicals (Bishop et al., “Physiological roles of amyloid-beta and implications for its removal in Alzheimer's disease” Drugs Aging 21:621-630 (2004); and iii) improvement of neuronal survival in tissue culture (Plant et al., “The production of amyloid beta peptide is a critical requirement for the viability of central neurons” J Neurosci 23:5531-5535 (2003).

It has also been reported that in the brain of patients with AD, the clearance of Aβ is decreased. Mawuenyega et al., “Decreased clearance of CNS beta-amyloid in Alzheimer's disease” Science 330:1774 (2010). Although the precise causes of decreased Aβ clearance are not known, the diminution of vascular transport is a probable contributor to the accumulation of Aβ. Zlokovic B. V., “Neurovascular mechanisms of Alzheimer's neurodegeneration” Trends Neurosci 28:202-208 (2005). The “hallmark” appearance of Aβ in the brain of patients with AD therefore may represent a “downstream” (i.e., secondary) result of microvascular attenuation. The loss of neurons and synapses, and resulting impairment of cognitive function, are consequences of the impaired blood flow and decreased trophic effect of microvascular endothelium.

The reported inverse association of cancer and AD may also be a result of reduced angiogenesis—an association suggesting a role of angiogenesis in cancer. Driver et al., “Inverse association between cancer and Alzheimer's disease: results from the Framingham Heart Study” Br Med J 344:e1442 (2012); Musicco et al., “Inverse occurrence of cancer and Alzheimer disease: a population-based incidence study” Neurology 81:322-328 (2013); and Folkman J., “Angiogenesis: an organizing principle for drug discovery? Nat Rev Drug Discov 6:273-286 (2007).

Impaired microvascular integrity—whether caused by age and vascular disease-related attenuation, the Notch—vascular loss-of-function model, or a combination of both—explains more of the apparently dissonant features of AD than the widely subscribed β-amyloid toxic gain-of-function model. In both microvascular attenuation mechanisms, β-amyloid increases as a downstream consequence. Cerebral degeneration results from loss of microvascular endothelial trophic function and decreased cerebral blood flow. Increased β-amyloid is then a secondary marker, and neither removal of Aβ nor interference with γ-secretase would be expected to improve cognition or to prevent further decline of cerebral structure or function. Of other β-amyloid-related strategies, the inhibition of β-secretase might be successful by preventing the formation of soluble (s)APPb from APP, thereby decreasing the competition of APP with Notch for cleavage by PS.

### F. Conclusions

The presence of β-amyloid-containing neuritic plaques has been a hallmark pathological change of AD and regarded by those in the art as central to its diagnosis. Although β-amyloid is associated with AD, the evidence that it is causally related is modest, and has eroded over time, as data regarding its specific, unique, and invariable spatial and temporal relation to the development and neural mechanisms causing LOSAD are elusive and increasingly negative. Although Aβ cannot be proved to have no causal relation to the pathogenesis of dementia in AD—the null hypothesis—it is increasingly likely that Aβ is merely a biomarker, a later consequence of upstream changes that lead to neuronal and synaptic losses. When microvascular failure and decreased Notch-related angiogenesis occur in the age-related setting of multicausation vulnerability of the brain, the accelerated neural and cognitive decline of AD occurs.

In one embodiment, present invention contemplates a microvascular mechanism having clear therapeutic implications for AD. Strategies based on removal of Aβ, intending to alter the course of the disease, are likely to continue to fail. Multiple intrinsic and extrinsic age-related factors erode the integrity of the elderly brain, and may be somewhat modified by the prevention of long-standing losses resulting from hypertension, trauma, drugs, and so on. To prevent LOS AD, and the catastrophic decline in function and structure, however, it is best to consider the upstream causes of the decline. Improvement of cerebral microvascular endothelial function may ameliorate the precipitating cause of AD. Facilitating Notch-1 function, or decreasing the substrate competition of APP or sAPPb with Notch, for cleavage by PS/γ-secretase is likely to modify the disease successfully. Strategies that improve microvascular function, increase cerebral angiogenesis, or facilitate Notch cleavage may ameliorate degenerative changes seen in AD, and constitute an effective disease-modifying strategy.

### III. Induction of Cerebral Microvessel Angiogenesis in AD Patients

Early in the course of AD—as an initial event—synaptic, axonal, then neuronal loss, with atrophy of brain in affected areas, is believed to take place. While a decline of many molecular housekeeping functions may contribute to this involutional atrophic change, which eventually evolves into major, widespread cortical atrophy, degeneration and dementia, this degenerative cascade and atrophy may be initiated by a loss of microvascular trophic and nutritional functions. In one embodiment, the present invention contemplates a therapeutic intervention comprising improving brain microvascular endothelial function. While direct monitoring of trophic functions of the endothelium is not practical, microvascular integrity and function can be assessed by measuring changes in cerebral blood flow; the paracrine and microvascular flow functions are parallel, related functions. Assessing cognitive function should also provide information on the important beneficial effects on brain function.

The data presented herein shows the results of a clinical study with AD patients, where cognitive function was monitored over an initial four month treatment period, and then subsequently followed-up over a timespan ranging from months to several years. Adverse events attributable to the medications used were not reported.

MRI-based arterial spin labeling and gadolinium perfusion studies revealed a significant degree of increase in cerebral blood flow from baseline to the conclusion of the study period. Psychometric studies revealed improvements in psychometric test performance, subjective measures of cognition and performance of activities of daily living as reported by the patients, their spouses, and family members. Such psychometric tests include, but are not limited to, Clinical Dementia Rating (CDR) and/or Clinicians Global Impression of Change (CIBIC Plus)).

One goal of this study was to determine whether patients diagnosed with mild Alzheimer's disease or Mild Cognitive Impairment (MCI) would respond with increased cerebral blood flow and improved cognitive function when treated with a combination of drugs that increase endothelial nitric oxide synthase (eNOS). Decreased cerebral blood flow is characteristic of AD and it has been shown that brain microvascular endothelium in AD patients is scanty and poorly stained immunohistochemically. It has also been shown that statin drugs reduce the incidence of AD in individuals treated with statins, independent of cholesterol levels. It is further known that microvascular endothelium functions as a paracrine organ having trophic secretory activity. Consequently, microvascular endothelium may play a role in maintaining normal function and growth of cells, including neurons, and the healthy maintenance of organs. Because microvascular capillaries are anatomically positioned in direct proximity to every one of the hundred billion neurons in the brain, it is believed that endothelial trophic secretions, as well as blood supply to neurons, may play a role brain survival and/or function.

Although it is not necessary to understand the mechanism of an invention it is believed that the method described herein comprise a plurality of synergistic combination treatment regimens improve endothelial function. For example, a synergistic combination treatment comprises simvastatin, L-arginine, and sapropterin (Kuvan). Although it is not necessary to understand the mechanism of an invention, it is believed that the synergistic combination treatment improves endothelial nitric oxide synthase (eNOS) activity. eNOS activity may be a surrogate measure of microvascular endothelial function thereby providing a measure of treatment efficacy.

Cerebral blood flow (CBF) was evaluated using MRI-based arterial spin labeling and contrast perfusion flow by 3T MRI (i.e., for example, Siemens 3T Magnetom Trio). In one embodiment, patients diagnosed with AD were treated sequentially and cumulatively with a combined treatment regimen including simvastatin, L-arginine, and/or tetrahydrobiopterin. In one embodiment, the combined treatment paradigm improved microvascular and endothelial function. These data show that cerebral blood flow is increased subsequent to the administration of this combined treatment regimen and a majority of the AD patients showed improvement of cognitive function, while others showed stabilization of cognitive function.

Endothelial function can be facilitated by either increasing endothelial nitric oxide synthase (eNOS) activity or by reducing endothelin-1. Endothelial nitric oxide synthase, or Type 3 nitric oxide synthase, synthesizes NO from L-arginine, which then facilitates vascular relaxation via cGMP. eNOS activity can be increased by the administration of HMGCoA reductase inhibitors (i.e., for example, statins), which are widely used to reduce cholesterol synthesis by blocking the mevalonate pathway. Statins have pleiotropic effects, however, which are known to reduce the risk of vascular events well beyond the reduction of total cholesterol, or of LDL. The effects of statins on eNOS, and on endothelin-1, as well as their overall anti-inflammatory effects, are believed to be instrumental in its efficacy. Fujii et al., “Statins restore ischemic limb blood flow in diabetic microangiopathy via eNOS/NO upregulation but not via PDGF-BB expression” Am J Physiol Heart Ore Physiol 294:H2785-2791 (2008); Enomoto et al., “Rosuvastatin prevents endothelial cell death and reduces atherosclerotic lesion formation in ApoE-deficient mice” Biomed Pharmacother 63:19-26 (2009); and Strey et al., “Short-term statin treatment improves endothelial function and neurohormonal imbalance in normocholesterolaemic patients with non-ischaemic heart failure” Heart 92:1603-1609 (2006).

Production of NO can be enhanced by the administration of its precursor amino acid, L-arginine. Settergren et al., “1-Arginine and tetrahydrobiopterin protects against ischemia/reperfusion-induced endothelial dysfunction in patients with type 2 diabetes mellitus and coronary artery disease” Atherosclerosis 204(1):73-78 (2009). The synthesis of NO also requires a cofactor, tetrahydrobiopterin (e.g., BH4; sapropterin; Kuvan). Katusic et al., “Vascular protection by tetrahydrobiopterin: progress and therapeutic prospects” Trends Pharmacol Sci 30:48-54 (2009). In one embodiment, the present invention contemplates a method for stimulating brain eNOS activity by a stepwise addition of statins, L-Arginine and BH4. Although it is not necessary to understand the mechanism of an invention it is believed that a combined treatment regimen of statins, L-arginine and BH4 could counteract age-, environmental- and genetic factors impairing cerebral microvascular function, and enhance trophic and nutritional influences of brain microvascular function.

### IV. Medication Treatment Regimens

### A. Statins

Statins are generally known as a class of drugs that inhibit the enzyme HMG-CoA reductase which plays a central role in the production of cholesterol. High cholesterol levels have been associated with cardiovascular disease (CVD). Lewington et al., “Blood cholesterol and vascular mortality by age, sex, and blood pressure: a meta-analysis of individual data from 61 prospective studies with 55,000 vascular deaths” Lancet 370(9602): 1829-1839 (2007). Statins have been found to reduce cardiovascular disease and mortality in those who are at high risk. The evidence is strong that statins are effective for treating CVD in the early stages of a disease (secondary prevention) and in those at elevated risk but without CVD (primary prevention). Taylor et al., “Statins for the primary prevention of cardiovascular disease” Cochrane Database Syst Rev. 1:CD004816 (2013). Side effects of statins include, but are not limited to, muscle pain/damage, increased risk of diabetes mellitus and/or liver enzyme abnormalities Naci et al., “Comparative Tolerability And Harms Of Individual Statins: A Study-Level Network Meta-Analysis Of 246,955 Participants From 135 Randomized, Controlled Trials” Circ Cardiovasc Qual Outcomes 6(4): 390-399 (2013); and Abd et al., “Statin-induced myopathy: a review and update.” Expert Opinion On Drug Safety” 10(3): 373-387 (2011).

A number of statins FDA-approved including, but not limited to, atorvastatin, fluvastatin, lovastatin, pitavastatin, pravastatin, rosuvastatin and/or simvastatin. Sweetman, S. C., “Cardiovascular Drugs”. In. Martindale: The Complete Drug Reference (36th ed.). London: Pharmaceutical Press, pp. 1155-1434 (2009). Several combination preparations of a statin and another agent, such as ezetimibe/simvastatin, are also available.

There are reports of cognitive decline with statins, however, a systematic review by the Canadian Working Group Consensus Conference concluded that the available evidence is not strongly supportive of a major adverse effect of statins. McDonagh, J. “Statin-Related Cognitive Impairment in the Real World: You'll Live Longer, But You Might Not Like It”. JAMA Intern Med. 174:1889 (2014); and Mancini et al., “Diagnosis, prevention, and management of statin adverse effects and intolerance: Canadian Working Group Consensus update” Can J Cardiol 29(12): 1553-1568 (2013). Another meta-analysis report concluded that the evidence suggests that there is no increase in dementia, mild cognitive impairment or reduction in cognitive performance scores as a result of statin administration. However, high doses have not been fully evaluated. Richardson et al., “Statins And Cognitive Function: A Systematic Review” Ann Intern Med. 159(10):688-697 (2013). Nonetheless, the FDA has required a warning regarding a possible relationship between statins and memory loss, forgetfulness and confusion, or other possible cognitive impacts, fda.gov/ForConsumersConsumerUpdales/ucm293330.htm; and accessdata.fda.gov.

Statins, by inhibiting the HMG CoA reductase pathway, inhibit the production of specific prenylated proteins. This inhibitory effect on protein prenylation may be involved, at least partially, in the improvement of endothelial function, modulation of immune function, and other pleiotropic cardiovascular benefits of statins, Lahera et al., “Endothelial Dysfunction, Oxidative Stress And Inflammation In Atherosclerosis: Beneficial Effects Of Statins” Curr Med Chem 14(2): 243-248 (2007); Blum et al., “The Pleiotropic Effects Of Statins On Endothelial Function, Vascular Inflammation, Immunomodulation And Thrombogenesis” Atherosclerosis 203(2): 325-330 (2009); and Porter et al., “Statins And Myocardial Remodeling: Cell And Molecular Pathways” Expert Rev Mol Med 13:e22 (2011).

As noted above, statins exhibit action beyond lipid-lowering activity in the prevention of atherosclerosis. The ASTEROID trial showed direct ultrasound evidence of atheroma regression during statin therapy. Nissen et al., “Effect Of Very High-Intensity Statin Therapy On Regression Of Coronary Atherosclerosis: The ASTEROID Trial” JAMA 295(13): 1556-1565 (2006). The data suggest that statins may have multiple physiological effects, including but not limited to, improving endothelial function, modulating inflammatory responses, maintaining plaque stability and/or preventing thrombus formation. Furberg C. D., “Natural Statins And Stroke Risk”. Circulation 99(2):185-188 (1999).

In one embodiment, the present invention contemplates a therapeutic regimen comprising simvastatin. Simvastatin can produce elevation of liver function tests in approximately 1% of patients, and myalgia in about 1-2%, with elevated creatine phosphokinase (CPK) in less than 2%. Rarely, rhabdomyolysis (muscle breakdown) may occur, evidenced by muscle pain, weakness and elevated CPK. Additional uncommon side effects include GI irritation, constipation, skin rash, allergic reactions, or hematologic reactions, headache, upper respiratory infection, renal failure and tendon rupture.

### B. Nitric Oxide Synthase Substrates

Nitric oxide synthases (EC 1.14.13.39) (NOSs) are a family of enzymes that are generally believed to catalyze the production of nitric oxide (NO) from L-arginine. NO may then function as a cellular signaling molecule. Although it is not necessary to understand the mechanism of an invention, NO is believed to modulate physiological functions including, but not limited to, vascular tone, insulin secretion, airway tone, peristalsis, angiogenesis and/or neural development. It may also function as a retrograde neurotransmitter. Nitric oxide is mediated in mammals by a calcium-calmodulin controlled isoenzyme termed endothelial nitric oxide synthase (eNOS) and/or neuronal nitric oxide synthase (nNOS). NOS catalyzes the reaction.

L-arginine+3/2 NADPH+1/2 H⁺2 O₂→citrulline+nitric oxide+2H₂O+3/2 NADP+

NOS isoforms catalyze other leak and side reactions, such as superoxide production at the expense of NADPH. As such, this stoichiometry is not generally observed, and reflects the three electrons supplied per NO by NADPH. NOSs may bind with a plurality of different cofactors. In general, the electron flow in the NO synthase reaction may be mediated by: NADPH→FAD→FMN→heme→O₂. Tetrahydrobiopterin also provides an additional electron during the catalytic cycle which is replaced during turnover. NOS is the only known enzyme that binds flavin adenine dinucleotide (FAD), flavin mononucleotide (FMN), heme, tetrahydrobiopterin (BH4) and calmodulin.

NOS enzymes may exist as homodimers. In eukaryotes, for example, each monomer may have two major regions: an N-terminal oxygenase domain, which belongs to the class of heme-thiolate proteins, and a multi-domain C-terminal reductase, which is homologous to NADPH:cytochrome P450 reductase (EC 1.6.2.4) and other flavoproteins. The FMN binding domain is homologous to flavodoxins, and the two domain fragment containing the FAD and NADPH binding sites is homologous to flavodoxin-NADPH reductases. The interdomain linker between the oxygenase and reductase domains contains a calmodulin-binding sequence. The oxygenase domain is a unique extended beta sheet cage with binding sites for heme and pterin.

NOSs can be dimeric, calmodulin-dependent or calmodulin-containing cytochrome p450-like hemoprotein that combines reductase and oxygenase catalytic domains in one dimer, bear both flavin adenine dinucleotide (FAD) and flavin mononucleotide (FMN), and carry out a 5-electron oxidation of non-aromatic amino acid arginine with the aid of tetrahydrobiopterin. Chinje et al., “Role Of Nitric Oxide In Growth Of Solid Tumours: A Balancing Act” Essays Biochem. 32:61-72 (1997).

All three isoforms (e.g. eNOS, nNOS and iNOS), each of which is presumed to function as a homodimer during activation, share a carboxyl-terminal reductase domain homologous to the cytochrome P450 reductase. They also share an amino-terminal oxygenase domain containing a heme prosthetic group, which is linked in the middle of the protein to a calmodulin-binding domain. Binding of calmodulin appears to act as a “molecular switch” to enable electron flow from flavin prosthetic groups in the reductase domain to heme. This facilitates the conversion of O₂ and L-arginine to NO and L-citrulline. The oxygenase domain of each NOS isoform also contains an BH4 prosthetic group, which is required for the efficient generation of NO. Unlike other enzymes where BH4 is used as a source of reducing equivalents and is recycled by dihydrobiopterin reductase (EC 1.5.1.33), BH4 activates heme-bound O₂ by donating a single electron, which is then recaptured to enable nitric oxide release.

In certain cases of endothelial dysfunction, l-arginine becomes rate-limiting for NOS activity in spite of sufficiently high plasma l-arginine concentrations. It has been demonstrated that eNOS can obtain its substrate from the conversion of l-citrulline to l-arginine and from protein breakdown. In particular, eNOS activity was fully restored by supplementing either 1-citrulline or 1-arginine-containing dipeptides. Consequently, l-citrulline and 1-arginine-containing peptides derived from either intracellular protein breakdown or from the extracellular space may be eNOS substrate sources. Karbach et al., “Relative Contribution Of Different 1-Arginine Sources To The Substrate Supply Of Endothelial Nitric Oxide Synthase” J Mol Cell Cardiol. 51(5) 855-861 (2011).

NO may be formed from L-arginine by isoforms of nitric oxide synthase (NOS) via NG-hydroxy-L-arginine, with L-citrulline as a byproduct. Modulation of NO may be achieved by manipulating these NOS substrates. While omission of L-arginine significantly reduced NOS phenotype, a partial compensation for this L-arginine withdrawal was achieved with L-citrulline and NG-hydroxy-L-arginine. Mitchell et al., “Expression Of Nitric Oxide Synthase And Effect Of Substrate Manipulation Of The Nitric Oxide Pathway In Mouse Ovarian Follicles” Hum Reprod. 19(1):30-40 (2004).

L-arginine is an amino acid normally present in the diet. It is not a prescription item, and is sold OTC. Most adverse effects occur only with parenteral injection of L-Arginine in large doses. Adverse effects are uncommon, and can include GI symptoms, including stomach discomfort, diarrhea, nausea, vomiting, bloating and abdominal cramps; renal toxicity, including elevated BUN and creatinine levels, cutaneous flushing; hypotension, endocrine effects including release of growth hormone, insulin, glucagon and prolactin; numbness and headache.

### C. Biopterins

Biopterins are pterin derivatives which function as endogenous enzyme cofactors in many species of animals and in some bacteria and fungi. Biopterins act as cofactors for aromatic amino acid hydroxylases (AAAH), which are involved in the synthesis a number of neurotransmitters including, but not limited to, dopamine, norepinephrine, epinephrine, and serotonin, along with several trace amines. Furthermore, nitric oxide synthase (NOS also utilizes biopterin derivatives as cofactors. In humans, tetrahydrobiopterin is an endogenous cofactor for AAAH enzymes.

Biopterin compounds found within a mammal include, but are not limited to, BH4 and BH2. Tetrahydrobiopterin, also referred to as BH4, THB, Kuvan® or sapropterin, is a naturally occurring cofactor of aromatic amino acid hydroxylase enzymes, and is a cofactor for the production of nitric oxide (NO) by the nitric oxide synthases. Chemically, its structure is that of a reduced pteridine derivative. As BH4 serves as a catalyst for the production of nitric oxide the compound modulates vasodilation, which improves systematic blood flow. A deficiency of BH4—and thus, of nitric oxide—has been proposed as a cause of the neurovascular dysfunction in circulation-related diseases such as diabetes. Wu et al., “Nitric oxide and vascular insulin resistance” BioFactors 35(1): 21-27 (2009). Dihydrobiopterin (BH2) is a pteridine compound produced in the synthesis of dopa, dopamine, norepinephrine and epinephrine. It is restored to the required cofactor tetrahydrobiopterin by dihydrobiopterin reductase.

Sapropterin is a commercially available synthetic preparation of the dihydrochloride salt of naturally occurring tetrahydrobiopterin (Kuvan®. 6R-BH4 or BH4). It can produce gastrointestinal side effects, including nausea (4%), abdominal pain (5%), diarrhea (8%) and vomiting (8%); headache (15%); and upper respiratory symptoms—nasal discharge (11%), pain in throat (10%), and upper respiratory infection (12-17%). Gastritis, spinal cord injury, streptococcal infection, testicular cancer and urinary tract infection have occurred with Kuvan treatment, but were considered not caused by BH4.

### V. Neurodegenerative Diseases

Neurodegenerative diseases are usually associated with a progressive loss of structure or function of neurons, including death of neurons. For example, many neurodegenerative diseases are known including, but not limited to, amyotrophic lateral sclerosis, multiple sclerosis, Parkinson's disease, Alzheimer's disease, dementias and/or Huntington's disease. For the most part, such diseases are presently considered incurable, resulting in progressive degeneration and/or death of neuron cells. Neurodegeneration can be found in many different levels of neuronal circuitry ranging from molecular to systemic.

### A. Alzheimer's Disease

Alzheimer's disease is usually characterized by loss of neurons and synapses in the cerebral cortex and certain subcortical regions. This loss results in gross atrophy of the affected regions, including degeneration in the temporal lobe and parietal lobe, and parts of the frontal cortex and cingulate gyrus. Wenk G. L., “Neuropathologic changes in Alzheimer's disease” J Clin Psychiatry 64 Suppl 9:7-10 (2003).

As discussed in detail herein, Alzheimer's disease has been (mis)hypothesized by many to be a protein misfolding disease (proteopathy), caused by accumulation of abnormally folded A-beta and tau proteins in the brain. Hashimoto et al., “Role of protein aggregation in mitochondrial dysfunction and neurodegeneration in Alzheimer's and Parkinson's diseases” Neuromolecular Med. 4(1-2):21-36 (2003). Plaques are believed to be made up of small peptides, 39-43 amino acids in length, called beta-amyloid (also written as A-beta or Aβ). Beta-amyloid is a fragment from a larger protein called amyloid precursor protein (APP), a transmembrane protein that penetrates through the neuron's membrane. APP has been associated with neuron growth, survival and post-injury repair. Priller et al., “Synapse formation and function is modulated by the amyloid precursor protein” J. Neurosci. 26 (27): 7212-7221 (2006), and Turner et al., “Roles of amyloid precursor protein and its fragments in regulating neural activity, plasticity and memory” Prog. Neurobiol. 70(1): 1-32 (2003). In Alzheimer's disease, an unknown process causes APP to be divided into smaller fragments by enzymes through proteolysis. Hooper N. M., “Roles of proteolysis and lipid rafts in the processing of the amyloid precursor protein and prion protein” Biochem. Soc. Trans. 33(Pt 2):335-338 (2005). One of these fragments gives rise to fibrils of beta-amyloid, which form clumps that deposit outside neurons in dense formations known as senile plaques. Tiraboschi et al., “The importance of neuritic plaques and tangles to the development and evolution of AD” Neurology 62(11): 1984-9 (2004), and Ohnishi et al., “Amyloid fibrils from the viewpoint of protein folding” Cell. Mol. Life Sci. 61(5):511-524 (2004).

### B. Dementia

Dementia is often used interchangeably with Alzheimer's disease, however, these two conditions are considered by many to be subject to differential diagnosis. Also known as senility, dementia may include a broad category of brain diseases that cause a long term and often gradual decrease in the ability to think and remember that is great enough to affect a person's daily functioning. Other common symptoms include emotional problems, problems with language, and a decrease in motivation. Diagnosis of a dementia usually involves an observed change from a person's usual mental functioning and a greater decline than one would expect due to aging.

Common types of dementia may include, but are not limited to, vascular dementia (25%), Lewy body dementia (15%), and/or frontotemporal dementia. Recently, the DSM-5 reclassified dementia as a neurocognitive disorder, with various degrees of severity. Association, American Psychiatric, In; Diagnostic And Statistical Manual Of Mental Disorders: DSM-5. (5th ed.). Washington, D.C.: American Psychiatric Association, pp. 591-603 (2013). Diagnosis is usually based on history of the illness and cognitive testing with medical imaging and blood work used to rule out other possible causes.

One form of pre-dementia is Mild Cognitive Impairment (MCI). MCI is usually associated with a person that exhibits memory or thinking difficulties, but not severe enough yet for a dementia diagnoses. MCI patients usually score between 25-30 on an MMSE. Usually, approximately 70% of people with MCI go on to develop some form of dementia. MCI is generally divided into two categories. The first is one that is primarily memory loss (amnestic MCI). The second category is anything that is not primarily memory difficulties (non-amnestic MCI). People with primarily memory problems generally go on to develop Alzheimer's disease. People with the other type of MCI may go on to develop other types of dementia.

Diagnosis of MCI is often difficult, as cognitive testing may be normal. Often, more in-depth neuropsychological testing is necessary to make the diagnosis, the most commonly used criteria are called the Peterson criteria and include, but are not limited to: i) memory or other cognitive (thought-processing) complaint by the person or a person who knows the patient well; ii) a memory or other cognitive problem as compared to a person of the same age and level of education; iii) a problem not severe enough to affect the person's daily function; and/or iv) no current diagnosis of a dementia.

### C. Parkinson's Disease

Parkinson's disease (PD) is believed to be a degenerative disorder of the central nervous system. It results from the death of dopamine-generating cells in the substantia nigra, a region of the midbrain; the cause of cell-death is unknown. PD is generally known to manifest symptoms including, but not limited to, bradykinesia, rigidity, resting tremor and/or posture instability. A prevalence rate of PD has been reported to range from 15 per 100,000 to 12,500 per 100,000, and the incidence of PD from 15 per 100,000 to 328 per 100,000, with the disease being less common in Asian countries.

The main known risk factor is age. Susceptibility genes include, but are not limited to, α-synuclein, leucine-rich repeat kinase 2 (LRRK-2), and glucocerebrosidase (GBA) have shown that genetic predisposition is another important causal factor. However, one mechanism by which the brain cells in Parkinson's are lost may include an abnormal accumulation of the protein alpha-synuclein bound to ubiquitin in the damaged cells. The alpha-synuclein-ubiquitin complex cannot be directed to the proteosome. This protein accumulation forms proteinaceous cytoplasmic inclusions called Lewy bodies. The latest research on pathogenesis of disease has shown that the death of dopaminergic neurons by alpha-synuclein may be due to a defect in the machinery that transports proteins between two major cellular organelles—the endoplasmic reticulum (ER) and the Golgi apparatus. Certain proteins like Rab1 may reverse this defect caused by alpha-synuclein in animal models.

Recent research suggests that impaired axonal transport of alpha-synuclein leads to its accumulation in the Lewy bodies. Experiments have revealed reduced transport rates of both wild-type and two familial Parkinson's disease-associated mutant alpha-synucleins through axons of cultured neurons. De Vos et al., “Role of axonal transport in neurodegenerative diseases” Annual Review of Neuroscience 31:151-173 ((2008). Membrane damage by alpha-synuclein could be another Parkinson's disease mechanism. Varkey et al., “Membrane curvature induction and tubulation are common features of synucleins and apolipoproteins” The Journal of Biological Chemistry 285(42):32486-32493 (2010).

### D. Huntington's Disease

Huntington's disease (HD) is believed to causes astrogliosis and/or a loss of medium spiny neurons. Lobsiger et al., “Glial cells as intrinsic components of non-cell autonomous neurodegenerative disease” Nat. Neurosci. 10(11): 1355-1360 (2007); Purves et al., “Modulation of Movement by the Basal Ganglia—Circuits within the Basal Ganglia System”. In: Dale Purves. Neuroscience (2nd ed.). Sunderland, Mass.: Sinauer Associates. ISBN 0-87893-742-0 (2001); and Estrada Sánchez et al., “Excitotoxic neuronal death and the pathogenesis of Huntington's disease” Arch. Med. Res. 39(3):265-276 (2008). Areas of the brain are affected according to their structure and the types of neurons they contain, reducing in size as they cumulatively lose cells. The areas affected are mainly in the striatum, but also the frontal and temporal cortices. The striatum's subthalamic nuclei send control signals to the globus pallidus, which initiates and modulates motion. The weaker signals from subthalamic nuclei thus cause reduced initiation and modulation of movement, resulting in the characteristic movements of the disorder. Crossman A. R., “Functional anatomy of movement disorders” J. Anat. 196(4).519-525 (2000). Mutant Huntingtin is an aggregate-prone protein. During the cells' natural clearance process, these proteins are retrogradely transported to the cell body for destruction by lysosomes. It is a possibility that these mutant protein aggregates damage the retrograde transport of important cargoes such as BDNF by damaging molecular motors as well as microtubules.

### E. Amyotrophic Lateral Sclerosis (ALS)

Amyotrophic lateral sclerosis (ALS or Lou Gehrig's Disease) is a disease in which motor neurons are selectively targeted for degeneration. In 1993, missense mutations in the gene encoding the antioxidant enzyme Cu/Zn superoxide dismutase 1 (SOD1) were discovered in subsets of patients with familial ALS. This discovery led researchers to focus on unlocking the mechanisms for SOD1-mediated diseases. However, the pathogenic mechanism underlying SOD1 mutant toxicity has yet to be resolved. More recently, TDP-43 and FUS protein aggregates have been implicated in some cases of the disease, and a mutation in chromosome 9 (C9orf72) is thought to be the most common known cause of sporadic ALS.

Recent research has provided in vitro evidence that the primary cellular sites where SOD1 mutations act are located on astrocytes. Astrocytes may then cause the toxic effects on the motor neurons. The specific mechanism of toxicity still needs to be investigated, but the findings are significant because they implicate cells other than neuron cells in neurodegeneration. Nagai et al., “Astrocytes expressing ALS-linked mutated SOD1 release factors selectively toxic to motor neurons” Nature Neuroscience 10(5):615-622 (2007), Di Giorgio et al., “Non-cell autonomous effect of glia on motor neurons in an embryonic stem cell-based ALS model” Nature Neuroscience 10(5):608-614 (2007); and Julien J. P., “ALS: astrocytes move in as deadly neighbors” Nature Neuroscience 10(5) 535-537 (2007).

### F. Multiple Sclerosis

Multiple sclerosis (MS), also known as disseminated sclerosis or encephalomyelitis disseminata, is a demyelinating disease in which the insulating covers of nerve cells in the brain and spinal cord are damaged. This damage disrupts the ability of parts of the nervous system to communicate, resulting in a wide range of signs and symptoms including but not limited to, physical, mental and sometimes psychiatric problems. Compston et al., “Multiple sclerosis” Lancet 372(9648). 1502-1517 (2008); and Compston et al., “Multiple sclerosis” Lancet 359(9313): 1221-1231 (2002). MS usually presents as one of two types with new symptoms either, i) occurring in isolated attacks (e.g., remitting/relapsing MS); or ii) building up over time (e.g., progressive MS). Between attacks, symptoms may disappear completely; however, permanent neurological problems often occur, especially as the disease advances. Reingold S. C., “Defining the clinical course of multiple sclerosis: results of an international survey” Neurology 46(4):907-911 (1996).

While the cause of MS is not clear, the underlying mechanism is thought to be either destruction by the immune system or failure of the myelin-producing cells. Nakahara et al., “Current concepts in multiple sclerosis: autoimmunity versus oligodendrogliopathy” Clinical Reviews In Allergy & Immunology 42(1):26-34 (2012). Proposed causes for this include genetics and environmental factors such as infections. Ascherio et al., “Environmental risk factors for multiple sclerosis. Part I: the role of infection” Annals of Neurology 61(4):288-299 ((2007).

### VI. Pharmaceutical Compositions and Formulations

The present invention further provides pharmaceutical compositions (e.g., comprising the compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carriers) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product. Agents that enhance uptake of oligonucleotides at the cellular level may also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), also enhance the cellular uptake of oligonucleotides.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual oligonucleotides, and can generally be estimated based on EC50s found to be effective in in vitro and in vivo animal models or based on the examples described herein. In general, dosage is from 0.01 μg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example I

### Clinical Study Design

The present study design represents a clinical study with AD patients, where cognitive function was monitored over an initial four month treatment period, and then subsequently followed-up over a timespan ranging from months to several years. Adverse events attributable to the medications used are recorded and evaluated.

MRI-based arterial spin labeling and gadolinium perfusion studies evaluate cerebral blood flow beginning at a patient baseline level and over the entire treatment period. Psychometric studies to evaluate cognitive parameters include, but are not limited to psychometric test performance, subjective measures of cognition and performance of activities of daily living as reported by the patients, their spouses, and family members. Such psychometric tests include, but are not limited to, Clinical Dementia Rating (CDR) and/or Clinicians Global Impression of Change (CIBIC Plus)).

### Inclusion Criteria

Subjects range in age between 55-85 and exhibit mild Alzheimer's Disease or Mild Cognitive Impairment (MCI) and a Mini-Mental State Examination (MMSE) score ranging between 15-26. Subjects also have a caregiver who can provide information, and bring patient to the sessions; no known allergies to any of the medications to be used; normal renal function; willingness of patient and spouse/responsible caregiver to participate.

### Exclusion Criteria

Patient cannot have:
i) a significant diagnosed psychiatric disorder;
ii) suffered from a stroke;
iii) current use of any of the test medications (e.g., for example, a statin, L-arginine, Kuvan®);
iv) suffer from phenylketonuria (PKU);
v) exhibit an elevated serum phenylalanine level (>10 mg/dL);
vi) an allergy to any of the test medications;
vii) a current active malignancy;
viii) a renal insufficiency as determined by an elevated creatinine of above 1.3 mg/dl;
ix) exhibit an abnormal liver function (e.g., for example, at least a doubled alanine aminotransferase (ALT) or aspartate aminotransferase (AST) level above normal),
x) exhibit a serious disease including, but not limited to, coronary insufficiency, congestive heart failure, carotid stenosis greater than 50%, active peptic ulcer, urinary tract or other active infection, cancer (except skin cancer, or 5 years inactive breast or prostate cancer); pregnancy, or
xi) an inability to attend follow-up evaluations.

Subjects may continue to take anticholinesterase drugs for Alzheimer's Disease (i.e., for example, Aricept®, Exelon®, Razadyne®) and/or Namenda®, if they have been on the drug(s) for at least 3 months. Subjects on levodopa and male subjects taking drugs for erectile dysfunction (Viagra®, Cialis®, Levitra®) are cautioned regarding hypotension.

One primary endpoint of the study was an increase of cerebral blood flow CBF) resulting from a combination of three (3) experimental drugs, comparing the CBF at the end of the study with the baseline determination. Global CBF and focal CBF were evaluated and compared over several brain areas including, but not limited to, hippocampus; parietal lobes and/or frontal lobes.

One secondary endpoint of the study was improved cognitive performance of the subjects on the psychometric tests over the course of the combined treatment regimen and at the end of the 4-month study which was compared with each patient's baseline performance. These psychometric tests include, but are not limited to, the Mini-Mental State Examination (MMSE), Alzheimer's Disease Assessment Scale-Cognitive (ADAS-Cog) test, Cognitive Assessment Screening Test (CAST), the Clinical Dementia Rating (CDR/sum of boxes) test, and the Clinician Interview-Based Impression of Change (CIBIC) test and/or Caregiver evaluations.

Safety endpoints that may terminate a patient testing participation include, but are not limited to, significant adverse symptoms, abnormal observations on physical examination and/or any significant adverse changes clinical monitoring parameters including, but not limited to, complete blood count (CBC), metabolic profile, lipid profile, creatinine phosphokinase (CPK), urine analysis (UA) and/or electrocardiogram (EKG).

As of the filing date of this application, data is still being collected and/or processed in accordance with this study.

### Example II

### Neurological Examinations

The neurological examinations performed herein include, but are not limited to, a general physical examination or a Cognitive Assessment Screening Test (CAST).

### Example III

### Clinical Monitoring Parameters

The clinical monitoring parameters performed herein include, but are not limited to, a complete blood count (CBC), a differential blood count (DBC), erythrocyte sedimentation rate (ESR); a metabolic profile, c-reactive protein (CRP); a lipid profile, creatinine phosphokinase (CPK), free thyroxine index (FTI), thyroid stimulating hormone (TSH), anti-thyroid antibody, rapid plasma reagin (RPR); vitamin B₁₂; hemoglobin A_{1C}, antinuclear antibody (ANA), prothrombin international normalized ratio (PT/INR), and/or a urine analysis.

Test procedures to measure all of the above parameters are either well known in the art and/or commercial kits are available.

### Example III

### Neurological Imaging Scans

The neurological imaging scans performed herein include, but are not limited to, brain magnetic resonance imaging (MRI) brain magnetic resonance angiogram imaging (MRA) and/or neck MRA imaging. MRI and MRA scans may be performed without moving the patient by using different settings on the MRI scanner. MRA scanning may be performed following an intravenous injection of gadolinium. Claustrophobic patients may be given a tranquilizer (e.g., for example, Klonopin®, 0.5-1.9 mg/patient) to reduce anxiety during the scanning procedure.

### Example IV

### Cerebral Blood Flow Measurements

Cerebral blood flow (CBF) is performed by conventionally known MRI techniques. Claustrophobic patients may be given a tranquilizer (e.g., for example, Klonopin®, 0.5-1.9 mg/patient) to reduce anxiety during the scanning procedure.

### Example V

### Psychometric Testing

Psychometric tests performed herein include, but are not limited to, Mini-Mental State Examination (MMSE), Alzheimer's Disease Assessment Scale-Cognitive (ADAS-Cog) test, Cognitive Assessment Screening Test (CAST), the Clinical Dementia Rating (CDR/sum of boxes) test, and the Clinician Interview-Based Impression of Change (CIBIC) test.

### Example VI

### Drug Administration And Testing Timeline

Three (3) FDA-approved drugs were used, sequentially and cumulatively, including simvastatin, L-arginine and sapropterin (tetrahydrobiopterin). In addition, patients who have significant claustrophobia during MRI scans may receive a small oral dose (0.5 to 1.0 mg) of clonazepam (Klonopin®).

### Drug Administration:

1. Treatment Regimen I: Weeks 1-4 (i.e., for example, one month)
a. Simvastatin 40 mg/day

2. Treatment Regimen II: Weeks 5-8 (i.e., for example, one month)
a. Simvastatin 40 mg/day+L-Arginine 2.0 gm/TID

3. Treatment Regimen III: Weeks 9-16 (i.e., for example, two months)
a. Simvastatin 40 mg/day+L-Arginine 2.0 gm/TID+sapropterin 20 mg/kg/day

Following the completion of each Treatment Regimen the following cognitive function tests and clinical monitoring tests were administered;
1) Adverse effects reported by caregiver and/or patient including, but not limited to, dizziness, headache, confusion, sleep disturbance, rash, cardiac symptoms, respiratory symptoms, GI symptoms, urinary symptoms; other (per informant)
2) CBC, metabolic profile, lipid profile, CPK, and UA.
3) MMSE, CIBIC Plus and Caregiver information.
4) Cerebral blood flow imaging
   The next Treatment Regimen was implemented when all laboratory tests are within acceptable normal limits. At the end of the study (e.g., Week 16) the following tests were given:
   1) Adverse effects reported by caregiver and/or patient including, but not limited to, dizziness, headache, confusion, sleep disturbance, rash, cardiac symptoms, respiratory symptoms, GI symptoms, urinary symptoms; other (per informant)
   2) CBC, metabolic profile, lipid profile, CPK, UA, EKG
   3) MMSE; ADAS-Cog; CDR/sum of boxes and CIBIC plus Caregiver information
   4) Cerebral blood flow imaging

As of the filing date of this application, data is still being collected and/or processed in accordance with this study.

### Example VII

### Data Analysis

Cerebral blood flow and psychometric data obtained for each individual subject at baseline were compared with any changes observed in comparable data obtained at each subsequent assessment following Treatment Regimen I, Treatment Regimen II and Treatment Regimen III. The data can be analyzed to determine change: improvement, stability or decline; and to assess statistical significance of magnitude of change with addition of each added drug (and the conjoint administration of all three drugs) during the four-month course of the study.

I claim:

1. A method, comprising:
a) providing;
i) a patient exhibiting at least one symptom of a neurodegenerative disease; and
ii) a pharmaceutical composition comprising a statin; and
b) administering said pharmaceutical composition to said patient under conditions such that said at least one symptom is reduced.

2. The method of claim 1, wherein said pharmaceutical composition further comprises a nitric oxide synthase substrate.

3. The method of claim 1, wherein said pharmaceutical composition further comprises a biopterin compound.

4. The method of claim 1, wherein said pharmaceutical composition comprises a combination of said statin and said nitric oxide synthase substrate.

5. The method of claim 1, wherein said pharmaceutical composition comprises a combination of said statin, said nitric oxide synthase substrate and said biopterin compound.

6. The method of claim 1, wherein said statin pharmaceutical composition is administered for a first time period.

7. The method of claim 4, wherein said combined statin/nitric oxide substrate pharmaceutical composition is administered for a second time period.

8. The method of claim 5, wherein said combined statin/nitric oxide substrate/biopterin compound composition is administered for a third time period.

9. The method of claim 6, wherein said first time period precedes said second time period.

10. The method of claim 7, wherein said second time period precedes said third time period.

11. The method of claim 6, wherein said first time period is one month.

12. The method of claim 7, wherein said second time period is one month.

13. The method of claim 8, wherein said third time period is two months.

14. The method of claim 1, wherein said statin is selected from the group consisting of atorvastatin, fluvastatin, lovastatin, pitavastatin, pravastatin, rosuvastatin and simvastatin.

15. The method of claim 2, wherein said nitric oxide substrate is selected from the group consisting of L-arginine, L-citrulline and NG-hydroxy-L-arginine.

16. The method of claim 3, wherein said biopterin is selected from the group consisting of tetrahydrobiopterin and dihydrobipterin.

17. The method of claim 1, wherein said at least one symptom comprises reduced cognitive function.

18. The method of claim 1, wherein said at least one symptom comprises cerebral atrophy.

19. The method of claim 1, wherein said at least one symptom comprises reduced brain microvascular endothelial function.

20. The method of claim 4, said combined statin/nitric oxide substrate pharmaceutical compound is synergistic as compared to said statin pharmaceutical compound.

21. The method of claim 5, wherein said combined statin/nitric oxide substrate/biopterin compound pharmaceutical compound is synergistic as compared to said statin pharmaceutical compound.

22. The method of claim 5, wherein said combined statin/nitric oxide substrate/biopterin compound pharmaceutical compound is synergistic as compared to said combined statin/nitric oxide substrate pharmaceutical compound.

23. The method of claim 1, wherein said neurodegenerative disease is Alzheimer's disease.

24. The method of claim 1, wherein said neurodegenerative disease is dementia.

25. The method of claim 1, wherein said neurodegenerative disease is selected from the group consisting of Huntington's disease, amyotrophic lateral sclerosis, multiple sclerosis, and Parkinson's disease.

26. The method of claim 1, wherein said method further comprises a step of improving brain microvascular and endothelial function.

27. The method of claim 1, wherein said method further comprises a step of stimulating brain endothelial nitric oxide synthase activity.

28. The method of claim 1, wherein said neurodegenerative disease comprises an early stage Alzheimer's disease.

29. The method of claim 1, wherein said neurodegenerative disease comprises a mild cognitive impairment.

30. The method of claim 27, wherein said stimulated brain endothelial nitric oxide synthase activity improves brain microvascular endothelial function.
